(19) United States
(12) Patent Application Publication
(10) Pub. No.: US 2004/0210958 A1
(43) Pub. Date: Oct. 21, 2004

(54) A Novel Culture Method for Corn Transformation

(75) Inventors: David R. Duncan, St. Charles, MO (US); Vladimir Sidorov, Chesterfield, MO (US)

Correspondence Address: MONSANTO COMPANY, 800 N. LINDBERGH BLVD., ATTENTION: G.P. WUELLNER, IP PARALEGAL, (E2NA), ST. LOUIS, MO, 63167, US

(73) Assignee: MONSANTO TECHNOLOGY LLC, St. Louis, MO (US)

(21) Appl. No.: 10/708,724

(22) Filed: Mar. 19, 2004

Related U.S. Application Data
(60) Provisional application No. 60/320,022, filed on Mar. 19, 2003.

(51) Int. Cl. A01H 1/00; C12N 15/82

(52) U.S. Cl. 800/278

(57) ABSTRACT

The present invention relates to a novel culture system for generating transformed corn plants from mature seeds. In particular, the invention relates to the use of plant hormones during germination to affect the culture response. Transgenic corn plants can then be easily produced.

This application claims priority to US provisional application 60/320,022 filed Mar. 19, 2003, herein incorporated by reference in its entirety.

### BACKGROUND OF INVENTION

The present invention relates to the field of plant biotechnology. In particular, provided herein are systems for culturing corn zygotic embryos to produce callus and genetically transforming embryogenic cultures of corn derived therefrom.

It is now widely known that genes can be transferred from a wide range of organisms to crop plants by recombinant DNA technology. This advance has provided enormous opportunities to improve plant resistance to pests, disease and herbicides, and to modify biosynthetic processes to change the quality of plant products.

Microparticle- and Agrobacterium-mediated gene delivery are the two most commonly used plant transformation methods. U.S. Pat. No. 5,631,152 describes a rapid and efficient microprojectile bombardment method for the transformation and regeneration of monocots. Microparticle-mediated transformation refers to the delivery of DNA coated onto microparticles that are propelled into target tissues by several methods.

Agrobacterium-mediated transformation is achieved through the use of a genetically engineered soil bacterium belonging to the genus Agrobacterium. Several Agrobacterium species mediate the transfer of a specific DNA known as “T-DNA”, that can be genetically engineered to carry any desired piece of DNA into many plant species. The major events marking the process of T-DNA mediated pathogenesis are induction of virulence genes, and processing and transfer of T-DNA. This process is the subject of many reviews (e.g., Ream, Ann. Rev. Phytopathol. 27: 583-618, 1989; Howard and Citovsky, Bioassays, 12:103-108, 1990).

The major deficiencies in current plant transformation systems include but are not limited to the production efficiency of the system and transformation difficulties due to genotype or species diversity and explant limitations. U.S. Pat. No. 5,591,616 describes a method for transforming monocots that depends on the use of freshly cultured immature embryos or cultured immature embryos or callus. In either system, the explants must be freshly isolated, and the method is labor intensive, genotype-specific, and explant-limited. It takes considerable effort and resources to maintain donor plants and prepare the explants, and this limits flexibility in planning and implementing experiments, causing significant greenhouse and labor costs.

### SUMMARY OF INVENTION

The present invention provides novel methods for the stable and efficient transformation of plants using mature seeds as the starting material.

In one aspect the present invention provides a method of obtaining a transformable explant from a zygotic embryo in which the embryo is germinated in tissue culture media containing an effective amount of an auxin, with or without an effective amount of a cytokinin, to produce a plant, a nodal section is isolated from the plant and then cultured to produce embryogenic or meristematic cultures capable of being transformed.

More specifically, the invention provides a novel method of obtaining a transformable explant from a mature corn seed in which the seed is germinated in a tissue culture media containing an effective amount of an auxin, with or without an effective amount of a cytokinin, to produce a growing plant; isolating a nodal section from such growing plant; and culturing the nodal section to produce an embryogenic or a meristematic culture. This culture is then used as the explant for transformation and transformed tissue is regenerated to produce a transgenic plant.

Still another aspect of the present invention relates to transformed plants produced by producing a transformable cell or tissue from a mature corn seed germinated in tissue culture media containing an effective amount of an auxin and optionally an effective amount of a cytokinin, inoculating it with an Agrobacterium containing at least one genetic component capable of being transferred to the plant cell or tissue, selecting a transformed plant cell or tissue and regenerating a transformed plant expressing the genetic component from the selected plant cells or tissues.

Yet another aspect of the present invention relates to any seeds, or progeny of the transformed plants produced by the method of the present invention.

Further objects, advantages and aspects of the present invention will become apparent from the accompanying figures and description of the invention.

### BRIEF DESCRIPTION OF DRAWINGS

FIG. 1 is a plasmid map of pMON30113.

FIG. 2 is a plasmid map of pMON65375.

### DETAILED DESCRIPTION

The following definitions will aid in the understanding of the description of the invention.

“Callus” refers to a dedifferentiated proliferating mass of cells or tissue.

“Type I callus” refers to morphologically compact maize callus from which whole plants can be regenerated via organogenesis, embryogenesis or a combination of the two.

“Type II callus” refers to morphologically friable, highly embryogenic maize callus (Armstrong and Green, Planta, 164:207-214, 1985).

“Explant” refers to a plant part that is capable of being transformed and subsequently regenerated into a transgenic plant. Typical explants include immature embryos, callus, cotyledons, meristems, leaves, or stems.

“Mature seed” refers to a seed harvested from a plant and appropriately treated for long-term storage and capable of germination.

“Mature embryo” refers to a zygotic embryo approximately 15 days or more after pollination that cannot typically produce regenerable callus when cultured in vitro.

“Immature embryo” refers to a zygotic embryo approximately 15 days or less after pollination that can typically produce regenerable callus when cultured in vitro.

The term “zygotic embryo” is used to encompass mature embryos extracted from mature seed, mature embryos, or immature embryos extracted from immature seed and capable of germination.

“Embryogenic culture” refers to dedifferentiated cultured plant tissues that regenerate whole plants by passing through a developmental sequence similar to that of a zygote.

“Multiple bud culture” refers to a proliferating mass of plant tissues organized as a collection of structures similar in appearance to a shoot apical meristem.

“Nodal section” refers to an excised portion of a germinating seedling that contains the shoot apical meristem, all subtending axillary meristems and associated leaf base tissue.

“Plant growth regulators or plant hormones” refer to compounds that affect plant growth. The plant growth regulators include auxins, cytokinins, ABA, gibberellins, ethylene, brassinosteroids, and polyamines. Auxins affect the elongation of shoots and roots at low concentration but inhibit growth at higher levels. Commonly used auxins include picloram (4-amino-3,5,6-trichloropicolinic acid), 2,4-D (2,4-dichlorophenoxyacetic acid), IAA (indole-3-acetic acid), NAA (α-naphthaleneacetic acid), and dicamba (3,6-dichloroanisic acid). Cytokinins cause cell division, cell differentiation, and shoot differentiation. Commonly used cytokinins include kinetin, BA (6-benzylaminopurine), 2-ip (2-isopentenyladenine), BAP (6-benzylaminopurine), thidiazuron (TDZ), zeatin riboside, and zeatin.

“Tissue culture media” refers to liquid, semi-solid, or solid media used to support plant growth and development in a non-soil environment. Suitable plant tissue culture media is known to one of skill in the art, as discussed in detail subsequently.

“Coding sequence”, “coding region” or “open reading frame” refers to a region of continuous sequential nucleic acid triplets encoding a protein, polypeptide, or peptide sequence.

“Endogenous” refers to materials originating from within the organism or cell.

“Exogenous” refers to materials originating from outside of the organism or cell. It refers to nucleic acid molecules used in producing transformed or transgenic host cells and plants. As used herein, exogenous is intended to refer to any nucleic acid that is introduced into a recipient cell, regardless of whether a similar nucleic acid may already be present in such cell.

“Genome” refers to the chromosomal DNA of an organism. The genome is defined as a haploid set of chromosomes of a diploid species. For the purposes of this application, genome also includes the organellar genome.

“Monocot” or “monocotyledonous” refers to plants having a single cotyledon. Examples include cereals such as maize, rice, wheat, oat, and barley.

“Nucleic acid” refers to deoxyribonucleic acid (DNA) or ribonucleic acid (RNA).

“Phenotype” refers to a trait exhibited by an organism resulting from the interaction of genotype and environment.

“Polyadenylation signal” or “polyA signal” refers to a nucleic acid sequence located 3′ to a coding region that promotes the addition of adenylate nucleotides to the 3′ end of the mRNA transcribed from the coding region.

“Promoter” or “promoter region” refers to a nucleic acid sequence, usually found 5′ to a coding sequence, that controls expression of the coding sequence by controlling production of messenger RNA (mRNA) by providing the recognition site for RNA polymerase or other factors necessary for the start of transcription at the correct site.

“Recombinant nucleic acid vector” or “vector” refers to any agent such as a plasmid, cosmid, virus, autonomously replicating sequence, phage, or linear or circular single- or double-stranded DNA or RNA nucleotide segment, derived from any source, capable of genomic integration or autonomous replication, comprising a nucleic acid molecule in which one or more nucleic acid sequences have been linked in a functionally operative manner. Such recombinant nucleic acid vectors or constructs are capable of introducing a 5′ regulatory sequence or promoter region and a DNA sequence for a selected gene product into a cell in such a manner that the DNA sequence is transcribed into a functional mRNA, which is subsequently translated into a polypeptide or protein.

“Regeneration” refers to the process of growing a plant from a plant cell.

“Regenerable callus” refers to callus from which whole plants can be produced but where the mode of regeneration (embryogenesis or organogenesis) has not been determined or is not pertinent to the discussion.

“Selectable marker” or “screenable marker” refers to a nucleic acid sequence whose expression confers a phenotype facilitating identification of cells containing the nucleic acid sequence.

“Transcription” refers to the process of producing an RNA copy from a DNA template.

“Transformation” refers to a process of introducing an exogenous nucleic acid sequence (vector or construct) into a cell or protoplast, in which that exogenous nucleic acid is incorporated into the nuclear DNA, plastid DNA, or is capable of autonomous replication.

“Transgenic” refers to organisms into which an exogenous nucleic acid sequence has been integrated.

The present invention provides a method of obtaining transformable plant tissue from a mature seed or zygotic embryo by germinating it on media containing an effective amount of an auxin and, optionally, an effective amount of a cytokinin and then isolating the transformable tissue. The invention provides a transgenic plant resulting from this transformable tissue and a method for transformation of the transformable plant tissues of this invention and regeneration of the transformed cells or tissues into a differentiated transformed plant. The present invention finds particular utility in monocots and is particularly amenable for use with corn. The method greatly increases the efficiency of callus production.

The mature seed or zygotic embryo is germinated in a germination media containing an effective amount of an auxin with or without an effective amount of a cytokinin to produce a growing seedling containing a nodal section. A mixture of an auxin and a cytokinin gives the best response. The use of auxin or cytokinin alone is effective, but the combination is more effective in producing embryogenic callus from the nodal section. Thus, an effective amount of an auxin, a cytokinin, or a combination of an auxin and a cytokinin is an amount that permits the creation or development of embryogenic callus from the nodal section. Auxins affect the elongation of shoots and roots at low concentration but inhibit growth at higher levels. Commonly used auxins include picloram (4-amino-3,5,6-trichloropicolinic acid), 2,4-D (2,4-dichlorophenoxyacetic acid), IAA (indole-3-acetic acid), NAA (α-naphthaleneacetic acid), and dicamba (3,6-dichloroanisic acid). Cytokinins cause cell division, cell differentiation, and shoot differentiation. Commonly used cytokinins include kinetin, BA (6-benzylaminopurine), 2-ip (2-isopentenyladenine), BAP (6-benzylaminopurine), thidiazuron (TDZ), zeatin riboside, and zeatin. One of skill in the art can easily test combinations of auxins and cytokinins to arrive at alternative combinations. In the current invention, picloram and BAP are exemplified based on cost and performance. Also, 2,4-D is a preferred auxin. The concentration of auxin, for example picloram, could be from about 0.5 mg/L to about 20 mg/L or from about 1 mg/L to about 15 mg/L or from about 1 mg/L to about 10 mg/L. The concentration of cytokinin, for example BAP, could be from about 0.1 mg/L to about 10 mg/L or from about 0.5 mg/L to about 5 mg/L or from about 1 mg/L to about 3 mg/L. The ratio of auxin to cytokinin would not be expected to be the same across different pairs of compounds because of the differing activity levels of each compound. The ratio between auxin and cytokinins (with other phytohormones) in the plant tissue is thought to determine the developmental path the plant tissue will take. The combinations of auxin and cytokinins described in this invention are particularly useful for facilitating the induction of embryogenic callus from the apical and nodal regions of seedlings. One of skill in the art could predict reasonable concentrations of auxins and cytokinins that would work in the invention based on the knowledge of the potency of each compound. The presence of an effective amount of the auxin, and optionally an effective amount of the cytokinin, in the germination medium greatly increases the amount and quality of callus that is produced in the embryogenic cultures compared to previously reported methods.

The seeds may also be primed prior to germination. Seed priming can be done in many ways known to those of skill in the art. Typically, seeds are gas sterilized, then coated with wet clay and fungicide and incubated at about 28° C. for 2 days in the dark. Then the seeds are placed at 15° C. for 5 days in the dark, followed by 2 days at 23° C. or 28° C. in the light. The clay can be wet with water, which appears to be most efficient, or with the media used for germination. Priming promotes more uniform germination between seeds and enhances the callus induction of the isolated nodal sections.

Once the seeds or zygotic embryos have been germinated in germination media containing growth hormones as described above, nodal sections can be isolated from the growing seedlings for further use. Nodal sections can be obtained from about 3 days to about 30 days, with 7-10 days being a useful time frame. At 3 days, the nodal region is large enough to excise. After 7-10 days, the seedlings are about 3-4 cm long and easily handled. The portion of the seedling containing the coleoptile node and about 2-5 mm of subtending mesocotyl tissue and 2-5 mm of leaf tissue above the shoot apical meristem (about 0.5 cm) (all together comprising the nodal section) is cut and then split longitudinally. More callus response is obtained from the nodal section tissue as the seedling ages. After approximately 30 days, there is callus on the plant itself at the nodal region.

Isolated nodal sections are then placed on callus induction media. The appropriate callus induction media will depend upon the genotype and can be readily determined or ascertained by one skilled in the art. The callus induction media that works for callus induction of immature embryos in a genotype is typically suitable for pre-treated nodal sections. Any appropriate callus induction media can be used in the present invention. A portion of the induced callus will be incapable of regenerating plants, but a person skilled in the art of tissue culture can easily separate the callus types to produce a maintainable and regenerable callus useful in transformation or other tissue culture purposes (Duncan & Widholm, Plant Science, 61: 91-103,1989).

The regenerable material produced by the preceding can be used in any transformation protocol to produce transgenic plants.

To initiate a transformation process in accordance with the present invention, it is first necessary to select genetic components to be inserted into the plant cells or tissues. Genetic components can include any nucleic acid that is introduced into a plant cell or tissue using the method according to the invention. Genetic components can include non-plant DNA, plant DNA or synthetic DNA.

In a preferred embodiment, the genetic components are incorporated into a DNA composition such as a recombinant, double-stranded plasmid or vector molecule comprising at least one or more of following types of genetic components: (a) a promoter that functions in plant cells to cause the production of an RNA sequence, (b) a structural DNA sequence that causes the production of an RNA sequence that encodes a product of agronomic utility, and (c) a 3′ non-translated DNA sequence that functions in plant cells to cause the addition of polyadenylated nucleotides to the 3′ end of the RNA sequence.

The vector may contain a number of genetic components to facilitate transformation of the plant cell or tissue and regulate expression of the desired gene(s). In one preferred embodiment, the genetic components are oriented so as to express a mRNA, that in one embodiment can be translated into a protein. The expression of a plant structural coding sequence (a gene, cDNA, synthetic DNA, or other DNA) that exists in double-stranded form involves transcription of messenger RNA (mRNA) from one strand of the DNA by RNA polymerase enzyme and subsequent processing of the mRNA primary transcript inside the nucleus. This processing involves a 3′ non-translated region that adds polyadenylated nucleotides to the 3′ ends of the mRNA.

Means for preparing plasmids or vectors containing the desired genetic components are well known in the art. Vectors typically consist of a number of genetic components, including but not limited to regulatory elements such as promoters, leaders, introns, and terminator sequences. Regulatory elements are also referred to as cis- or trans-regulatory elements, depending on the proximity of the element to the sequences or gene(s) they control.

Transcription of DNA into mRNA is regulated by a region of DNA usually referred to as the “promoter”. The promoter region contains a sequence of bases that signals RNA polymerase to associate with the DNA and to initiate the transcription into mRNA using one of the DNA strands as a template to make a corresponding complementary strand of RNA.

A number of promoters that are active in plant cells have been described in the literature. Such promoters would include but are not limited to the nopaline synthase (NOS) and octopine synthase (OCS) promoters that are carried on tumor-inducing plasmids of Agrobacterium tumefaciens, the caulimovirus promoters such as the cauliflower mosaic virus (CaMV) 19S and 35S promoters and the figwort mosaic virus (FMV) 35S promoter, the enhanced CaMV35S promoter (e35S), the light-inducible promoter from the small subunit of ribulose bisphosphate carboxylase (ssRUBISCO, a very abundant plant polypeptide). All of these promoters have been used to create various types of DNA constructs that have been expressed in plants.

Promoter hybrids can also be constructed to enhance transcriptional activity (U.S. Pat. No. 5,106,739), or to combine desired transcriptional activity, inducibility and tissue specificity or developmental specificity. Promoters that function in plants include but are not limited to promoters that are inducible, viral, synthetic, constitutive as described, and temporally regulated, spatially regulated, and spatio-temporally regulated. Other promoters that are tissue-enhanced, tissue-specific, or developmentally regulated are also known in the art and envisioned to have utility in the practice of this invention.

Promoters may be obtained from a variety of sources such as plants and plant DNA viruses and include, but are not limited to, the CaMV35S and FMV35S promoters and promoters isolated from plant genes such as ssRUBISCO genes. As described below, it is preferred that the particular promoter selected should be capable of causing sufficient expression to result in the production of an effective amount of the gene product of interest.

The promoters used in the DNA constructs (i.e., chimeric/recombinant plant genes) of the present invention may be modified, if desired, to affect their control characteristics. Promoters can be derived by means of ligation with operator regions, random or controlled mutagenesis, etc. Furthermore, the promoters may be altered to contain multiple “enhancer sequences” to assist in elevating gene expression.

The mRNA produced by a DNA construct of the present invention may also contain a 5′ non-translated leader sequence. This sequence can be derived from the promoter selected to express the gene and can be specifically modified so as to increase translation of the mRNA. The 5′ non-translated regions can also be obtained from viral RNAs, from suitable eukaryotic genes, or from a synthetic gene sequence. Such “enhancer” sequences may be desirable to increase or alter the translational efficiency of the resultant mRNA. The present invention is not limited to constructs wherein the non-translated region is derived from both the 5′ non-translated sequence that accompanies the promoter sequence. Rather, the non-translated leader sequence can be derived from unrelated promoters or genes (see, for example U.S. Pat. No. 5,362,865). Other genetic components that serve to enhance expression or affect transcription or translational of a gene are also envisioned as genetic components.

The 3′ non-translated region of the chimeric constructs should contain a transcriptional terminator, or an element having equivalent function, and a polyadenylation signal that functions in plants to cause the addition of polyadenylated nucleotides to the 3′ end of the RNA. Examples of suitable 3′ regions are (1) the 3′ transcribed, non-translated regions containing the polyadenylation signal of Agrobacterium tumor-inducing (Ti) plasmid genes, such as the nopaline synthase (NOS) gene, and (2) plant genes such as the soybean storage protein genes and the small subunit of the ribulose-1,5-bisphosphate carboxylase (ssRUBISCO) gene. An example of a preferred 3′ region is that from the ssRUBISCO E9 gene from pea (European Patent Application 0385 962).

Typically, DNA sequences located a few hundred base pairs downstream of the polyadenylation site serve to terminate transcription. The DNA sequences are referred to herein as transcription-termination regions. The regions are required for efficient polyadenylation of transcribed messenger RNA (mRNA) and are known as 3′ nontranslated regions. RNA polymerase transcribes a coding DNA sequence through a site where polyadenylation occurs.

In one preferred embodiment, the vector contains a selectable, screenable, or scoreable marker gene. These genetic components are also referred to herein as functional genetic components, as they produce a product that serves a function in the identification of a transformed plant, or a product of agronomic utility. The DNA that serves as a selection device functions in a regenerable plant tissue to produce a compound that would confer upon the plant tissue resistance to an otherwise toxic compound. Genes of interest for use as a selectable, screenable, or scorable marker would include but are not limited to GUS, green fluorescent protein (GFP), luciferase (LUX), antibiotics like kanamycin (Dekeyser et al., Plant Physiol., 90:217-223, 1989), and herbicides like glyphosate (Della-Cioppa et al., Bio/Technology, 5:579-584, 1987). Other selection devices can also be implemented including but not limited to tolerance to phosphinothricin, bialaphos, and positive selection mechanisms and would still fall within the scope of the present invention.

The present invention can be used with any suitable plant transformation plasmid or vector containing a selectable or screenable marker and associated regulatory elements as described, along with one or more nucleic acids expressed in a manner sufficient to confer a particular desirable trait. Examples of suitable structural genes of agronomic interest envisioned by the present invention would include but are not limited to genes for insect or pest tolerance, herbicide tolerance, genes for quality improvements such as yield, nutritional enhancements, environmental or stress tolerances, or any desirable changes in plant physiology, growth, development, morphology or plant product(s).

Alternatively, the DNA coding sequences can effect these phenotypes by encoding a non-translatable RNA molecule that causes the targeted inhibition of expression of an endogenous gene, for example via antisense- or cosuppression-mediated mechanisms (see, for example, Bird et al., Biotech Gen. Engin. Rev., 9:207-227, 1991). The RNA could also be a catalytic RNA molecule (i.e., a ribozyme) engineered to cleave a desired endogenous mRNA product (see for example, Gibson and Shillitoe, Mol. Biotech. 7:125-137, 1997). More particularly, for a description of anti-sense regulation of gene expression in plant cells see U.S. Pat. No. 5,107,065 and for a description of gene suppression in plants by transcription of a dsRNA see U.S. Pat. No. 6,506,559, U.S. Patent Application Publication No. 2002/0168707 A1, and U.S. patent application Ser. No. 09/423,143 (see WO 98/53083), Ser. No. 09/127,735 (see WO 99/53050) and Ser. No. 09/084,942 (see WO 99/61631), all of which are incorporated herein by reference. Thus, any gene that produces a protein or mRNA that expresses a phenotype or morphology change of interest is useful for the practice of the present invention.

Exemplary nucleic acids that may be introduced by the methods encompassed by the present invention include, for example, DNA sequences or genes from another species, or even genes or sequences that originate with or are present in the same species, but are incorporated into recipient cells by genetic engineering methods rather than classical reproduction or breeding techniques. However, the term exogenous is also intended to refer to genes that are not normally present in the cell being transformed, or perhaps simply not present in the form, structure, etc., as found in the transforming DNA segment or gene, or genes that are normally present yet that one desires, e.g., to have over-expressed. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA that is already present in the plant cell, DNA from another plant, DNA from a different organism, or a DNA generated externally, such as a DNA sequence containing an antisense message of a gene, or a DNA sequence encoding a synthetic or modified version of a gene.

In light of this disclosure, numerous other possible selectable or screenable marker genes, regulatory elements, and other sequences of interest will be apparent to those of skill in the art. Therefore, the foregoing discussion is intended to be exemplary rather than exhaustive.

For Agrobacterium-mediated transformation, after the construction of the plant transformation vector or construct, said nucleic acid molecule, prepared as a DNA composition in vitro, is introduced into a suitable host such as E. coil and mated into another suitable host such as Agrobacterium, or directly transformed into competent Agrobacterium. These techniques are well-known to those of skill in the art and have been described for a number of plant systems including soybean, cotton, and wheat (see, for example U.S. Pat. Nos. 5,569,834 and 5,159,135, and WO 97/48814, herein incorporated by reference in their entirety).

The present invention encompasses the use of bacterial strains to introduce one or more genetic components into plants. Those of skill in the art would recognize the utility of Agrobacterium-mediated transformation methods. A number of wild-type and disarmed strains of Agrobacterium tumefaciens and Agrobacterium rhizogenes harboring Ti or Ri plasmids can be used for gene transfer into plants. Preferably, the Agrobacterium hosts contain disarmed Ti and Ri plasmids that do not contain the oncogenes that cause tumorigenesis or rhizogenesis, respectfully, which are used as the vectors and contain the genes of interest that are subsequently introduced into plants. Preferred strains would include but are not limited to Agrobacterium tumefaciens strain C58, a nopaline-type strain that is used to mediate the transfer of DNA into a plant cell, octopine-type strains such as LBA4404 or succinamopine-type strains, e.g., EHA101 or EHA105. The use of these strains for plant transformation has been reported and the methods are familiar to those of skill in the art.

The explants can be from a single genotype or from a combination of genotypes. Any corn seed that can germinate is a viable starting material. In a preferred embodiment, superior explants from plant hybrids can be used as explants. For example, a fast-growing cell line with a high culture response (higher frequency of embryogenic callus formation, growth rate, plant regeneration frequency, etc.) can be generated using hybrid embryos containing several genotypes. In a preferred embodiment, an F1 hybrid or first generation offspring of cross-breeding can be used as a donor plant and crossed with another genotype. Those of skill in the art are aware that heterosis, also referred to herein as “hybrid vigor”, occurs when two inbreds are crossed. The present invention thus encompasses the use of an explant resulting from a three-way cross, wherein at least one or more of the inbreds is highly regenerable and transformable, and the transformation and regeneration frequency of the three-way cross explant exceeds the frequencies of the inbreds individually. Other tissues are also envisioned to have utility in the practice of the present invention.

Any suitable plant culture medium can be used during the transformation process. Examples of suitable media would include but are not limited to MS-based media (Murashige and Skoog, Physiol. Plant, 15:473-497, 1962) or N6-based media (Chu et al., Scientia Sinica 18:659, 1975) supplemented with additional plant growth regulators including but not limited to auxins such as picloram (4-amino-3,5,6-trichloropicolinic acid), 2,4-D (2,4-dichlorophenoxyacetic acid) and dicamba (3,6-dichloroanisic acid); cytokinins such as BAP (6-benzylaminopurine) and kinetin; ABA; and gibberellins. Other media additives can include but are not limited to amino acids, macroelements, iron, microelements, inositol, vitamins and organics, carbohydrates, undefined media components such as casein hydrolysates, with or without an appropriate gelling agent such as a form of agar, such as a low melting point agarose or Gelrite® if desired. Those of skill in the art are familiar with the variety of tissue culture media, which when supplemented appropriately, support plant tissue growth and development and are suitable for plant transformation and regeneration. These tissue culture media can either be purchased as a commercial preparation or custom prepared and modified. Examples of such media would include but are not limited to Murashige and Skoog (Murashige and Skoog, Physiol. Plant, 15:473-497, 1962), N6 (Chu et al., Scientia Sinica 18:659, 1975), Linsmaier and Skoog (Linsmaier and Skoog, Physio. Plant., 18: 100, 1965), Uchimiya and Murashige (Uchimiya and Murashige, Plant Physiol. 15:473, 1962), Gamborg″s media (Gamborg et al., Exp. Cell Res., 50:151, 1968), D medium (Duncan et al., Planta, 165:322-332, 1985), McCown″s Woody plant media (McCown and Lloyd, HortScience 16:453, 1981), Nitsch and Nitsch (Nitsch and Nitsch, Science 163:85-87, 1969), and Schenk and Hildebrandt (Schenk and Hildebrandt, Can. J. Bot. 50:199-204, 1972) or derivations of these media supplemented accordingly. Those of skill in the art are aware that media and media supplements such as nutrients and growth regulators for use in transformation and regeneration and other culture conditions such as light intensity during incubation, pH, and incubation temperatures can be optimized for the particular variety of interest.

Once the transformable plant tissue is isolated, the next step of the method is introducing the genetic components into the plant tissue. This process is also referred to herein as “transformation.” The plant cells are transformed and each independently transformed plant cell is selected. The independent transformants are referred to as transgenic events. A number of methods have been reported and can be used to insert genetic components into transformable plant tissue. Microparticle- and Agrobacterium-mediated gene delivery are the two most commonly used plant transformation methods, but other methods are known.

Those of skill in the art are aware of the typical steps in the plant transformation process. Those of skill in the art are familiar with procedures for growth and suitable culture conditions for Agrobacterium as well as subsequent inoculation procedures. The density of the Agrobacterium culture used for inoculation and the ratio of Agrobacterium cells to explant can vary from one system to the next, and therefore optimization of these parameters for any transformation method is expected.

The next stage of the transformation process is the inoculation. In this stage the explants and Agrobacterium cell suspensions are mixed together. The duration and condition of the inoculation and Agrobacterium cell density will vary depending on the plant transformation system.

After inoculation, any excess Agrobacterium suspension can be removed and the Agrobacterium and target plant material are co-cultured. The co-culture refers to the time post-inoculation and prior to transfer to a delay or selection medium. Any number of plant tissue culture media can be used for the co-culture step. Plant tissues after inoculation with Agrobacterium can be cultured in a liquid or a semi-solid media. The co-culture is typically performed for about one to three days at a temperature of about 18° C.-30° C. The co-culture can be performed in the light or in light-limiting conditions. Lighting conditions can be optimized for each plant system as is known to those of skill in the art.

After co-culture with Agrobacterium, the explants typically can be placed directly onto selective media. Alternatively, after co-culture with Agrobacterium, the explants could be placed on media without the selective agent and subsequently placed onto selective media. Those of skill in the art are aware of the numerous modifications in selective regimes, media, and growth conditions that can be varied depending on the plant system and the selective agent. Typical selective agents include but are not limited to antibiotics such as geneticin (G418), kanamycin, paromomycin or other chemicals such as glyphosate. Additional appropriate media components can be added to the selection or delay medium to inhibit Agrobacterium growth. Such media components can include, but are not limited to, antibiotics such as carbenicillin or cefotaxime.

The cultures are subsequently transferred to a media suitable for the recovery of transformed plantlets. Those of skill in the art are aware of the number of methods to recover transformed plants. A variety of media and transfer requirements can be implemented and optimized for each plant system for plant transformation and recovery of transgenic plants. Consequently, such media and culture conditions disclosed in the present invention can be modified or substituted with nutritionally equivalent components, or similar processes for selection and recovery of transgenic events, and still fall within the scope of the present invention.

The transformants produced are subsequently analyzed to determine the presence or absence of a particular nucleic acid of interest contained on the transformation vector. Molecular analyses can include but is not limited to Southern blots (Southern, Mol. Biol., 98:503-517, 1975), or PCR (polymerase chain reaction) analyses, immunodiagnostic approaches, and field evaluations. These and other well known methods can be performed to confirm the stability of the transformed plants produced by the methods disclosed. These methods are well known to those of skill in the art and have been reported (see for example, Sambrook et al., Molecular Cloning, A Laboratory Manual, 1989).

Those of skill in the art will appreciate the many advantages of the methods and compositions provided by the present invention. The following examples are included to demonstrate the preferred embodiments of the invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments that are disclosed and still obtain a like or similar result without departing from the spirit and scope of the invention. All references cited herein are incorporated herein by reference to the extent that they supplement, explain, provide a background for, or teach methodology, techniques, or compositions employed herein.

### EXAMPLES

### Example 1

Seed Germination

Seeds of several different corn genotypes were used as initial starting material. Mainly dry or vapor-phase sterilization protocol was used for seed sterilization. Accordingly seeds were kept in a desiccator for 2-24 h with sterilizing gas, which was produced by mixing of 200 mL bleach (5.25 to 6.15% sodium hypochlorite) and 2 mL HCl. Seeds were also sterilized in 50% bleach for 20 min and washed with sterile water three times.

For germination, the kernels were inserted with the radicle end down into the medium. For germination either MS3 or MS3′ solid medium was used (Table 2) (MS3′ medium is CM4C Basal Phytagar medium with 1 mg/L BAP, 2.2 mg/L picloram and 100 mg/L ascorbic acid). Seeds were incubated in the light at 28° C. for 7-10 days.

The effect of media on germination of corn seeds after dry sterilization was studied. Different media typically used for embryogenic or organogenic cultures were tested (see media descriptions in Tables 1 and 2). On MS3, CM4C, CM4C+Ag, CM4C+Ag+ABA the seedlings grew slower; and root development was different from control (H₂O). On MS3 medium, the nodal area was expanded and no roots formed at the nodal region. This area with apical and adventitious meristem usually produced the regenerable callus.

TABLE 1
Media used in this invention

| Component | ½ MS VI | | ½ MS PL | | MS/BAP | | MSOD | |
|---|---|---|---|---|---|---|---|---|
| MS salts | 2.2 | g/L | 2.2 | g/L | 4.4 | g/L | 4.4 | g/L |
| Sucrose | 20 | g/L | 68.5 | g/L | 30 | g/L | — | |
| Maltose | — | | — | | — | | 40 | g/L |
| Glucose | 10 | g/L | 36 | g/L | — | | 20 | g/L |
| 1-Proline | 0.115 | g/L | 0.115 | g/L | 1.36 | g/L | — | |
| C asamino | — | | — | | 0.05 | g/L | — | |
| Acids | | | | | | | | |
| Glycine | 2 | mg/L | 2 | mg/L | — | | — | |
| 1-Asparagine | — | | — | | — | | 150 | mg/L |
| myo-Inositol | 100 | mg/L | 100 | mg/L | — | | 100 | mg/L |
| Nicotinic Acid | 0.5 | mg/L | 0.5 | mg/L | 0.65 | mg/L | 0.65 | mg/L |
| Pyridoxine HCl | 0.5 | mg/L | 0.5 | mg/L | 0.125 | mg/L | 0.125 | mg/L |
| Thiamine HCl | 0.1 | mg/L | 0.1 | mg/L | 0.125 | mg/L | 0.125 | mg/L |
| CaPantothenate | — | | — | | 0.125 | mg/L | 0.125 | mg/L |
| 2,4-D | — | | — | | 0.5 | mg/L | — | |
| Picloram | — | | — | | 2.2 | mg/L | — | |
| Silver Nitrate | — | | — | | — | | — | |
| Na-Thiosulfate | — | | — | | — | | — | |
| Phytagar | — | | — | | 7.0 | g/L | 7.0 | g/L |
| Low EEO | — | | — | | — | | — | |
| agarose | | | | | | | | |

TABLE 2
MS3, CM4C and their derivatives.

| Components | Amount/liter | | | | | | | | | | | | | |
|---|---|---|---|---|---|---|---|---|---|---|---|---|---|---|
| (stock conc.) | MS2 | | MS3 | | MSh⁻ | | CM4C | | CM4C + Ag | | MSV S34 | | MSW57 | |
| MS salts | 4.4 | g | 4.4 | g | 4.4 | g | 4.4 | g | 4.4 | g | 4.4 | g | 4.4 | g |
| MS vitamin 100x | 10 | mL | 10 | mL | 10 | ml | 10 | mL | 10 | mL | 10 | mL | 10 | mL |
| ThiamineHCl (0.4 mg/mL) | — | | — | | — | | — | | — | | — | | 1.25 | mL |
| Maltose | 40 | g | 40 | g | — | | 40 | g | 40 | g | 40 | g | — | |
| Casein Hydrolysate | 0.5 | g | 0.5 | g | — | | 0.1 | g | 0.1 | g | 0.1 | g | — | |
| Casamino Acids | — | | — | | — | | — | | — | | — | | 0.5 | g |
| MES | 1.95 | g | 1.95 | g | — | | 1.95 | g | 1.95 | g | 1.95 | g | — | |
| Magnesium Chloride | — | | — | | — | | 0.75 | g | 0.75 | g | 0.75 | g | — | |
| Sucrose | — | | — | | 30 | g | — | | — | | — | | 30 | g |
| Glutamine | — | | — | | — | | 0.5 | g | 0.5 | g | 0.5 | g | — | |
| L-Proline | — | | — | | — | | — | | — | | — | | 1.38 | g |
| Post Autoclave additives | | | | | | | | | | | | | | |
| 2,4-D (1 mg/ml) | 0.5 | | — | | — | | 0.5 | mL | 0.5 | mL | — | | 0.5 | mL |
| | 0.5 | mL | | | | | | | | | | | | |
| Picloram (1 mg/ml) | — | | 2.2 | mL | — | | 2.2 | mL | 2.2 | mL | 10 | mL | 2.2 | mL |
| BAP (0.5 mg/ml) | 4.0 | | 2.0 | mL | — | | — | | — | | 6 | mL | — | |
| | 4.0 | mL | | | | | | | | | | | | |
| Ascorbic Acid (50 mg/ml) | 2.0 | mL | 2.0 | mL | — | | 2 | mL | 2 | mL | 2 | mL | — | |
| Silver Nitrate (2 mg/ml) | — | | — | | — | | — | | 1.7 | mL | — | | 1.7 | mL |

* CM4C + Ag + ABA: The same as CM4C + Ag but also supplemented with 0.1 mg/L ABA.
— Adjust pH to 5.8 before autoclaving.
— Solidified with 7.0 g/L of Phytagar or 3.0 g/L Phytogel.

If the seedlings were grown on MS3 medium for about 2 weeks, they became completely abnormal with expanded nodal area. After removal of all leaves and subculture of the nodal area to fresh MS3 medium, meristematic tissue formed.

Thus, MS3 medium prevents normal development of plantlets and induced formation, in the nodal area, of a multiple bud culture. A pulse of MS3 medium during germination of seeds was beneficial for induction of regenerable callus, whether embryogenic or organogenic. Germination of seeds for a period of 7-10 days was the most suitable for induction of embryogenic or organogenic culture from the nodal region.

### Example 2

Production of Meristematic Cultures and Conversion to Embryogenic Cultures

Induction of green organogenic culture.

Seeds were dry sterilized and germinated on MS3 or MS2 medium in a Percival incubator (16-h light and 8-h dark, 27° C.) and 3- to 7-day-old seedlings were used for induction of organogenic cultures. A section (approximately 0.5 cm long) containing the nodal region (with apical meristem and leaf primordia) was dissected longitudinally and laid horizontally on meristem induction medium (MS3) with the wounded surface down. The apical meristem was identified, and the stem piece was completely removed above the apical meristem. If the cuttings were too long, the leaves started to grow and removing and trimming of leaves during culture was required. The plates were incubated with 16 h light photoperiod at 28° C. After 14 days of culture, the leaves were trimmed. The material was cultured for another 10-12 days at the same conditions. Usually at this time the organogenic (meristem) culture formed. The apical meristem and axillary bud were the origins of meristem culture. A whole piece of newly formed meristem/bud culture was detached and transferred to fresh MS3 medium at the same conditions.

In some media the primary shoots continued to elongate and were trimmed weekly. In another medium the meristem containing area quickly expanded; the meristematic regions enlarged and formed clusters of shoots and shoot primordia in approximately 4-6 weeks. The cultures were green, highly regenerable and considered organogenic.

Meristem cultures could be established from all genotypes tested. However the frequency of meristem induction and timeline for production of these culture was different for different genotypes. Usually meristem culture could be established in 2-3 months. The initiated meristem cultures from different genotypes were used in conversion experiments as initial material for production of embryogenic cultures. The established meristematic cultures could also be maintained by regular subculture on MS3 medium every 24 days for an extended period of time without losing regeneration ability and without accumulation of somaclonal variations.

Conversion of Green Meristem Cultures into Embryogenic Callus Cultures.

Meristem cultures from several corn genotypes were studied for their ability to be converted to embryogenic culture. The results of these experiments indicated that after 3-4 subcultures meristem cultures of most genotypes can be converted into typical embryogenic callus cultures. Again some genotypes responded better than others. Generally, after 2 weeks of culture the newly established meristem cultures can be used for conversion to embryogenic cultures.

In experiments with corn line 102 culture, the clumps of green organized structures were transferred to different media used for embryogenic cultures and incubated in the dark. After three subcultures on CM4C media the embryogenic cultures (Type I callus) were established. This callus was very similar to callus cultures originating from immature embryos. After the 102 calli were transferred to MSOD medium, shoots were initiated from most of the calli. The regeneration frequency from 102 was the same as from 104 embryogenic culture.

The 105 culture had a very good conversion response. The meristem culture of 105 was also maintained on MS3 medium. In conversion experiments with 105, the embryogenic callus started forming within a few weeks after transfer to callus induction medium

Typical Type I embryogenic culture was also obtained from meristem culture of corn line 103. But the conversion process was very slow. It took 4-6 transfer cycles; careful selection of the right callus was required during subcultures.

Some response was found with 101, which was cultured on different media used for callus induction and propagation. Unfortunately the media tested were not able to establish an embryogenic callus of 101 (Table 3).

From experiments with different genotypes it was found that CM4C medium supplemented with 0.1 mg/L ABA and 20 μM AgNO₃ was the most efficient for conversion and suitable for almost all studied genotypes (Table 3).

TABLE 3
Effect of media on conversion of meristem culture
into embryogenic culture.

| | | Response |
|---|---|---|
| | | (conversion of |
| Genotype | Medium* | meristem culture)** |
| 101 | CM4C | −−−− |
| | CM4C + ABA | −−++ |
| | CM4C + ABA + AgNO₃ | −−++ |
| | 15AA (0.5 mg/L 2,4-D, 2.2 mg/L Picl) | −−−+ |
| 102 | CM4C | ++++ |
| 103 | CM4C | −−−− |
| | CM4C + ABA | ++++ |
| | CM4C + ABA + AgNO₃ | ++++ |
| | 15AA (0.5 mg/L 2,4-D, 2.2 mg/L Picl) | −−−− |
| 104 | CM4C | −+++ |
| | CM4C + ABA | ++++ |
| | CM4C + ABA + AgNO₃ | ++++ |
| | 15AA (0.5 mg/L 2,4-D, 2.2 mg/L Picl) | −−−+ |
| 105 | CM4C | −+++ |
| | CM4C + ABA | ++++ |
| | CM4C + ABA + AgNO₃ | ++++ |
| | 15AA (0.5 mg/L 2,4-D, 2.2 mg/L Picl) | −+++ |

*ABA is 0.1 mg/L. AgNO₃ is 20 μM. Picl is picloram. 15AA found in Planta (1985) 165: 322-332.
**−−−− no formation of embryogenic cuture,
++++ formation of good embryogenic culture, Type I callus.

### Example 3

Direct Induction of Embryogenic Culture

Most of genotypes listed above have been studied for the direct induction from their seedlings of embryogenic callus. Several media, including MS3, CM4C, CM4C+Ag, CM4C+Ag+ABA, 15AA (0.5 mg/L 2,4-D+2.2 mg/L picloram) were tested for callus induction.

The effect of different sterilization procedures and different media used for germination on direct induction of callus was analyzed. The nodal area (˜0.5 cm long) of seedlings was isolated, cut longitudinally and placed on the media with the wounded side down on the callus (embryogenic) induction media. The cultures were incubated at 28° C. with a 16 h light photoperiod. After 3-4 weeks, the explants were subcultured onto fresh medium and incubated in the dark at 28° C. Calli were subcultured onto fresh medium every 3-4 weeks until enough material was produced for transformation.

It was demonstrated that the frequency of callus induction can depend not only on the medium used for culturing of nodal cuttings but also on the germination medium. The combination of seed sterilization method and medium used for germination can greatly influence the ability to produce callus. It was shown that frequency of callus initiation in corn line 107 was higher when dry sterilization and direct germination on meristem induction medium (MS3) was applied. Germination on MS3 medium was beneficial for induction of embryogenic callus for almost all genotypes. Only 104 formed callus at considerably high frequency when seeds were germinated on hormone-free medium. CM4C+ABA+Ag medium was the most suitable medium for callus initiation for most of the genotypes tested.

High callus induction frequency was obtained with 103 and KHI. But the callus was not completely embryogenic. It looked organogenic and derived from nodal axillary buds. After one subculture to the same medium (CM4C+ABA+Ag) nice Type-I callus from KHI was obtained. More subculture cycles were necessary for the establishment of good 103 callus. Two months were necessary for the initiation and establishment of KHI embryogenic callus suitable for transformation. And only after 3 months (from seed sterilization) could transformation experiments be conducted with 103 callus.

Sometimes the initial data did not always reflect the real frequency of callus production. Thus, initiation of 108 callus was good on CM4C+ABA+Ag or CM4C+0.8 mg/L 2,4-D+3 mg/L picloram medium but after several subculture cycles a good callus could not be recovered from most of the original calli. The callus of 108 could be initiated at high frequency but after subculture to the same medium the callus converts into non-embryogenic callus. Thus, for some genotypes the callus initiation medium was not suitable for maintenance of callus and additional research is required to perfect the callus maintenance medium.

Seed-derived type I callus of some genotypes could be converted into type II callus, similar to immature embryo-derived callus. 105 callus responds in this manner. After 2 subculture cycles on 15AA (Ag), type I callus obtained from nodal section of 7-day-old seedlings was completely converted to type II callus.

This is the first known demonstration of type II callus being produced from seed-derived tissue sources. Based on the data, many possible pathways for in vitro cultures of corn exist. The results of conversion of corn cultures open a new opportunity to manipulate corn cultures and produce desirable cultures using mature seeds as the initial source.

### Example 4

This example described experiments to determine the best combination of auxins and cytokinins in the germination tissue culture media. To improve the efficiency of callus induction from 106 seedlings, 36 different germination media were evaluated. Those media were MS-based with different combinations of picloram (0, 0.1, 1, 2.2, 5 or 10 mg/L) and BAP (0, 0.1, 1, 3, 5 or 10 mg/L) concentrations. The results from this medium screen indicated that a wide concentration range of picloram and BAP could be used to increase the frequency of callus induction from this genotype (Table 4), and all were better than the non-growth regulator containing media typically reported in the literature.

TABLE 4
The effect of seed germination medium on callus induction from
nodal section explants from 7-day-old seedlings (expressed as %
embryogenic calliper node).

| | 6-benzylamino purine (BAP, mg/L) | | | | | |
|---|---|---|---|---|---|---|
| Picloram (mg/L) | 0.00 | 0.10 | 1.00 | 3.00 | 5.00 | 10.00 |
| 0.00 | 0.00 | 4.76 | 0.00 | 14.29 | 0.00 | 0.00 |
| 0.10 | 0.00 | 9.52 | 0.00 | 0.00 | 4.76 | 0.00 |
| 1.00 | 9.52 | 14.29 | 14.29 | 4.76 | 9.52 | 4.76 |
| 2.20 | 9.52 | 42.86 | 28.57 | 14.29 | 9.52 | 4.76 |
| 5.00 | 19.05 | 14.29 | 47.62 | 38.10 | 14.29 | 19.05 |
| 10.00 | 4.76 | 28.57 | 38.10 | 23.81 | 23.81 | 4.76 |

Based on the callus induction rate from this first screen, 9 of the treatments (picloram at 2.2, 5 and 10 mg/L with BAP at 0.1, 1, and 3 mg/L) were considered to be superior to others. They were selected for further comparison. In the new experiment, corn line 106 dry seeds were sterilized with HCl/Clorox gas for 6 h and then germinated on the 9 different liquid media, each containing one combination of picloram and BAP levels. The nodal area (˜0.5 cm long) of 7-day-old seedlings was isolated, cut longitudinally and placed on the media with wounded side down on an MS-based medium containing 3.0 g/L phytagel, 1.38 g/L proline, 0.5 g/L casamino acids, an additional 5 mg/L thiamine HCl, 0.5 mg/L 2,4-D, 2.2 mg/L picloram and 3.4 mg/L silver nitrate. The cultures were incubated at 28° C. with a 16 h light photoperiod and examined weekly for 4 weeks. The meristem section was marked when its first callus tissue was visible. At the end of the fourth week, all the callus tissues were picked and transferred individually onto fresh callus induction medium for further growth. Sixteen days later, the callus type (embryogenic or organogenic) was identified. All the embryogenic and organogenic tissues from each treatment were collected separately and weighed to obtain the fresh weights.

As shown in Table 5, the meristem sections germinated on media containing 5 mg/L picloram+0.1 mg/L BAP, 10 mg/L picloram+1 mg/L BAP, and 10 mg/L picloram+3 mg/L BAP appeared to develop callus tissue faster than others with 26%, 30% and 25.9%, respectively, of the sections forming visible callus tissue in just one week on culture. The earlier response on these three treatments also resulted in the higher overall response rates at the end of four weeks (42%, 51.7% and 53.4%, respectively). This indicates that factors affecting the response speed also affect the final response rate. Although the results were not very conclusive in terms of the effect of the plant growth regulator level on the response speed and overall rate, higher picloram (5 or 10 mg/L) and BAP (1 or 3 mg/L) levels may be beneficial.

Because early developed embryogenic tissue is more useful for transformation, the treatments were also compared based on the number of meristem sections forming embryogenic tissues, and the amount of the tissue was determined 16 days after the tissues were separated from the meristem sections and cultured on fresh medium. From the results summarized in Table 6, although the same three treatments mentioned above also had more sections (11, 13 and 12, respectively) forming embryogenic tissue compared with others, the treatment with germination medium containing 2.2 mg/L picloram and 0.1 mg/L BAP produced the largest amount (0.8998 g) of embryogenic tissue from 8 sections. However, the treatment with 5 mg/L picloram and 0.1 mg/L BAP and the treatment with 10 mg/L picloram and 3 mg/L BAP produced the second and third largest amounts of embryogenic tissue, largely because they had more sections producing embryogenic tissue than most of the other treatments. It appears difficult to conclude which treatment is superior to others based on these results.

TABLE 5
Callus induction rate of corn line 106 meristem sections germinated on different
media for one week.

| Germ. medium² | | # sections | # sections forming callus (%) | | | | | | | | |
|---|---|---|---|---|---|---|---|---|---|---|---|
| Picl. | BAP | cultured¹ | 1 wk | | 2 wks | | 3 wks | | 4 wks | | Total |
| 2.2 | 0.1 | 60 | 6 | (10.0) | 7 | (11.7) | 6 | (10.0) | 1 | (1.7) | 20 (33.3) |
| 2.2 | 1.0 | 60 | 4 | (6.7) | 4 | (6.7) | 4 | (6.7) | 3 | (5.0) | 17 (28.3) |
| 2.2 | 3.0 | 60 | 8 | (13.3) | 2 | (3.3) | 2 | (3.3) | 1 | (1.7) | 13 (21.7) |
| 5.0 | 0.1 | 50 | 13 | (26.0) | 3 | (6.0) | 4 | (6.7) | 1 | (2.0) | 21 (42.0) |
| 5.0 | 1.0 | 54 | 7 | (13.0) | 5 | (9.3) | 1 | (1.8) | 0 | (0) | 13 (24.1) |
| 5.0 | 3.0 | 60 | 5 | (8.3) | 5 | (8.3) | 1 | (1.7) | 2 | (3.3) | 14 (23.3) |
| 10.0 | 0.1 | 60 | 3 | (5.0) | 3 | (5.0) | 5 | (8.3) | 6 | (10.0) | 18 (30.0) |
| 10.0 | 1.0 | 60 | 18 | (30.0) | 6 | (10.0) | 2 | (3.3) | 4 | (6.7) | 31 (51.7) |
| 10.0 | 3.0 | 58 | 15 | (25.9) | 11 | (19.0) | 5 | (8.3) | 0 | (0) | 31 (53.4) |

¹Two sections were dissected from each seedling.
²All the germination media are MS-based. They only differed from each other in the picloram and BAP levels as listed.

TABLE 6
Fresh weight (FW) of embryogenic tissue determined 16 days after
the tissues were separated from the meristem sections and cultured on
fresh medium.

| | FW of embryogenic tissue (g) |
|---|---|
| Germination medium¹ | From no. of |

| Picl. | BAP | sections | Total | Mean |
|---|---|---|---|---|
| 2.2 | 0.1 | 8 | 0.8998 | 0.1125 |
| 2.2 | 1.0 | 5 | 0.2094 | 0.0419 |
| 2.2 | 3.0 | 4 | 0.0443 | 0.0111 |
| 5.0 | 0.1 | 11 | 0.7984 | 0.0726 |
| 5.0 | 1.0 | 5 | 0.459 | 0.0918 |
| 5.0 | 3.0 | 5 | 0.1241 | 0.0249 |
| 10.0 | 0.1 | 3 | 0.1655 | 0.0552 |
| 10.0 | 1.0 | 13 | 0.7119 | 0.0548 |
| 10.0 | 3.0 | 12 | 0.3805 | 0.0317 |

¹All the germination media are MS-based. They only differed from each other in the picloram and BAP levels as listed.

### Example 5

Bacterial Strains and Plasmids

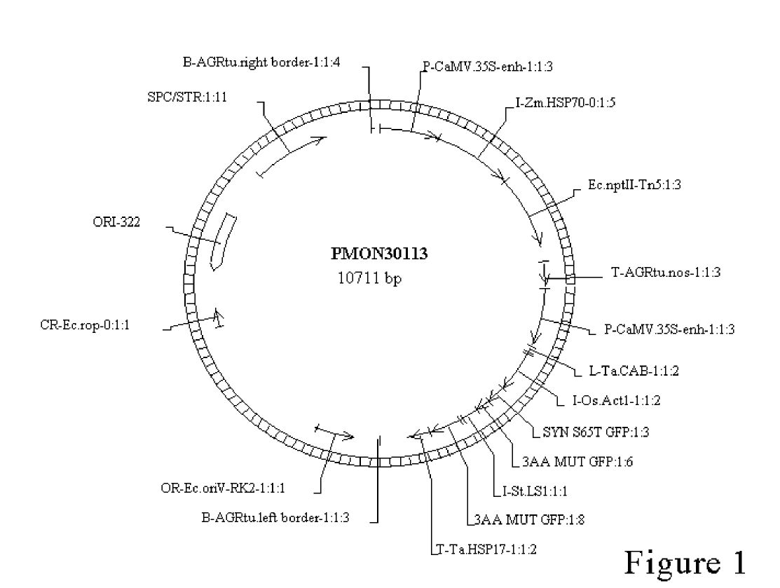

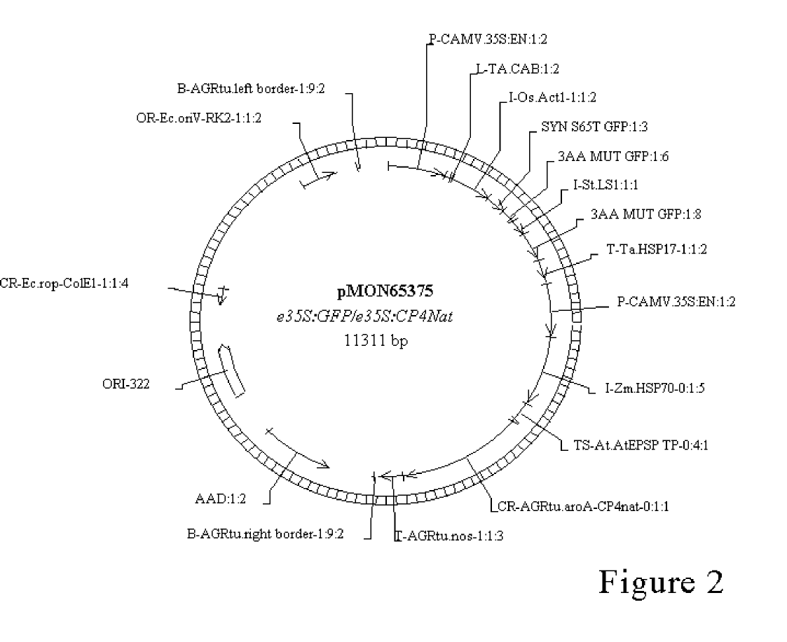

Agrobacterium tumefaciens strain ABI was harbored with a binary vector, pMON30113 (FIG. 1) or pMON65375 (FIG. 2). The T-DNA of the vectors contained a neomycin phosphotransferase II gene (nptII) or a EPSP synthase gene as the selectable marker and a green fluorescence protein gene (gfp) screenable marker, all driven by the 35S promoter.

### Example 6

Preparation of Agrobacterium

Agrobacterium ABI:pMON30113 or pMON65375 in glycerol stock were streaked out on solid LB medium supplemented with the antibiotics kanamycin (50 mg/L), spectinomycin (100 mg/L), streptomycin (100 mg/L) and chloramphenicol (25 mg/L) and incubated at 28° C. for 2 days. Two days before Agrobacterium inoculation, one colony from each Agrobacterium plate was picked up and inoculated into 25 mL of liquid LB medium supplemented with 100 mg/L of spectinomycin and 50 mg/L of kanamycin in a 250-mL flask. The flask was placed on a shaker at approximately 150 rpm and 27° C. overnight. The Agrobacterium culture was then diluted (1 to 5) in the same liquid medium and put back to the shaker. Several hours later in the late afternoon one day before inoculation, the Agrobacterium cells were spun down at 3500 rpm for 15 min. The bacterium cell pellet was re-suspended in induction broth with 200 μM of acetosyringone and 50 mg/L spectinomycin and 25 mg/L kanamycin, and the cell density is adjusted to 0.2 at O.D.₆₆₀. The bacterium cell culture (50 mL in each 250-mL flask) was then put back to the shaker and grown overnight. The following morning of inoculation day, the bacterium cells were spun down and washed with liquid ½MSVI medium (Table 1) supplemented with 200 μM of acetosyringone. After one more spinning, the bacterium cell pellet were re-suspended in 1/2MSPL medium (Table 1) with 200 μM of acetosyringone, and the cell density was adjusted to 1.0 at O.D.₆₆₀ for inoculation.

Reagents were commercially available and can be purchased from a number of suppliers (see, for example, Sigma Chemical Co., St. Louis, Mo.).

### Example 7

Agrobacterium-Mediated Transformation

Transformation of Embryogenic Callus Obtained from Seed-Derived Meristem Culture.

Embryogenic callus cultures of genotypes 102 and 103 that originate from established meristem cultures were used for Agrobacterium-mediated transformation. Cultures of both genotypes were maintained for a long time as meristem cultures and also as embryogenic cultures. Before transformation, 102 was maintained for about 7 months as a meristem culture and after conversion for 6 months as embryogenic culture. Corn line 103 meristem culture was maintained for 4 months and after conversion was subcultured every 3 weeks for another 4 months. High regeneration ability of both embryogenic lines was confirmed prior to transformation.

For transformation experiments, fresh cultures (5-8 days after subculture to new medium) were used. The Agrobacterium was prepared as described in Example 6. Individual calli from 3-5 mm were collected into an empty Petri plate. Fifteen to 20 mL of the Agrobacterium cell suspension were added to each plate, shaken, and set aside for 5 min. The Agrobacteria solution was removed with a pipette, then the calli were removed to a new plate containing Whatman #1 filter paper. The calli were spread out and the filters were moisturized with 200 μL of sterile water. The plates were sealed with parafilm and left overnight in the dark. The calli were then co-cultured with the Agrobacterium overnight in new petri dishes with 7-10 Whatman filters and 10 mL of liquid CM4C medium with 200 μL/L acetosyringone. The calli were then selected and regenerated as described in Example 8.

The results on Agrobacterium-mediated transformation of callus derived from established meristem cultures are presented in Table 7. The experiments demonstrated that embryogenic callus derived from meristem culture was suitable for transformation. The transformation frequency of these callus cultures was similar to callus derived from immature embryos. Transient and stable expression of GFP in 102 and 103 was demonstrated. The transformation efficiency ranged from 0 to 3.3% with 2%-3% being typical.

TABLE 7
Results on Agrobacterium-transformation of embryogenic callus obtained
from meristem culture [pMON30113 (NPTII/GFP)].

| # | | # Calli used | % GFP + calli | # GFP + calli | # Plants/ | TE |
|---|---|---|---|---|---|---|
| Exp. | Genotype | for tfn | (transient) | (stable) | events | % |
| Mar. 23, 2001 | 102 | 100 | ˜80 | 6 | 3 | 3 |
| Apr. 6, 2001 | 102 | 120 | ˜80 | 6 | 4 | 3.3 |
| | 102* | 80 | 0 | 0 | 0 | 0 |
| | 103** | 50 | ˜40 | 2 | 1 | 2 |
| May 8, 2001 | 103 | 100 | ˜50 | 3 | 3 | 3 |
| May 15, 2001 | 103 | 450 | ˜50 | 7 | 4 | 0.9 |

*The callus was growing on 15 AA medium containing 0.5 mg/L 2,4-D and 2.2 mg/L picloram.
**The callus was growing on CM4C + Ag + ABA.

Transformation of Embryogenic Callus Derived from Nodal Cuttings.

Embryogenic callus of several genotypes were used for Agrobacterium-mediated transformation. The callus was obtained directly from nodal cuttings of 6—10-day-old seedlings after germination of seeds on different media. All transformation experiments were conducted with a fresh callus (5-8 days after subculture to new medium) that was maintained in vitro for 2-4 months after initiation. The results of transformation experiments with this type of callus are presented in Tables 8 and 9. The stable expression of GFP was demonstrated in callus and regenerated plantlets. Transformation efficiency ranged from 0 to 6%, with some experiments being incomplete.

TABLE 8
Results on Agrobacterium-transformation of embryogenic callus obtained
from nodal cuttings [pMON30113 (NPTII/GFP)].

| # | | # Calli used | % GFP + calli | # GFP + calli | # Plants/ | TE |
|---|---|---|---|---|---|---|
| Exp. | Genotype | for tfn | (transient) | (stable) | events | % |
| Mar. 23, 2001 | 104 | 100 | ˜50 | 7 | 2 | 2 |
| May 8, 2001 | 104 | 100 | ˜80 | 20 | 6 | 6 |
| | 105 | 180 | ˜80 | 25 | 0 | 0 |
| Jul. 2, 2001 | 107 | 110 | ˜30 | 3 | (3)* | |
| Aug. 1, 2001 | 107 | 400 | ˜40 | 19 | (19)* | |

*Shoots were regenerated but not rooted.

TABLE 9
Results on Agrobacterium-transformation of
embryogenic callus obtained from
nodal cuttings of corn line 106/pMON65375 (EPSPS/GFP).

| | | | % putative |
|---|---|---|---|
| Construct | # callus | # events | Transformation |
| (pMON) | pieces | to soil | efficiency |
| 65375 | 711 | 24 + (5) | 4 |
| 65375 | 949 | 37 | 3.9 |
| Average | | | 4.0 |

### Example 8

After the co-cultivation, the callus pieces were transferred onto two pieces of 2 cm²×1 mm thick 100% acrylic felt with approximately 25 mL liquid CM4C medium (Table 2) supplemented with 750 mg/L carbenicillin and 100 mg/L paromomycin in petri dishes (100 mm×25 mm) with 20 to 25 embryos per plate. The plates were kept in a dark culture room at 28° C. for approximately 7-10 days. All the callus pieces were then transferred individually onto the solid CM4C medium supplemented with 750 mg/L carbenicillin and 100 mg/L paromomycin. The plates were kept in a dark culture room at 28° C. for approximately 4-6 weeks with one transfer. The cultures were then moved to a culture room with 16-h light/8-h dark photoperiod and 28° C. and placed on MS-6BA medium (Table 1) with 100 mg/L paromomycin and 500 mg/L carbenicillin. After 7 days, the callus pieces were transferred onto the second regeneration medium, a hormone-free MS-based medium (MSOD, Table 1) with 100 mg/L paromomycin in petri dishes (100 mm×25 mm). In another 2 weeks, the callus pieces that had shoots regenerated or were still alive were transferred onto the same hormone-free medium in Phytatrays for further growth. Regenerated plants (R) when they reached to the top of Phytatrays and had one or more healthy roots were moved to soil in peat pots in a growth chamber. In 7 to 10 days, they were transplanted into 12-in pots and moved to the greenhouse with conditions for normal corn plant growth. The plants were either self-pollinated or crossed with wild-type plants.

Glyphosate Selection and Regeneration on Liquid Medium

Culture plates with liquid medium, MSW57 (Table 2) with 500 mg/L carbenicillin and 0.1 mM glyphosate were prepared by placing 2 pieces of 2 cm²×1 mm thick felt squares in each Petri dish (100 mm×25 mm) and 25 mL of the medium. Callus pieces were transferred from the co-culture plates onto the first selection medium. During transfer, the callus pieces were separated into smaller callus pieces (2-3 mm). The plates were kept unsealed in plastic boxes at 27° C. in the dark for approximately 2 wks.

After the 2 weeks, as much of the 1^{st} selection medium as possible was removed by vacuum aspiration and replaced with 15-20 mL of the 2^{nd} selection medium, MSW57 with 500 mg/L carbenicillin and 0.25 mM glyphosate. The plates were kept under the same culture conditions for another 2 wks.

The 2^{nd} selection medium was removed by vacuum aspiration and replaced with 15-20 mL of MSBAP medium (Table 1) with 250 mg/L carbenicillin and 0.1 mM glyphosate. The plates were placed under a 16-h light regime at 27° C. for 5-7 days.

At the end of the BA pulse (MSBAP/C250/0.1 mM glyphosate medium), the BAP medium was replaced with 20 mL of MSOD medium (Table 1) with 250 mg/L carbenicillin and 0.1 mM glyphosate. All the dead callus pieces were removed, and the plates were kept under the same culture conditions with light. In approximately 2 wks on the regeneration medium, some callus pieces regenerated green shoots with roots. The healthy looking shoots were transferred into Phytatrays with MSOD/C250/0.1 mM glyphosate solidified with 3 g/L Phytagel. During transfer, callus tissue attached to the root area of the shoots was removed.

Move Regenerated Plants to Soil

When plants in Phytatrays reached the lid and developed one or more healthy roots (usually after 1-3 wks in Phytatrays), they were big enough to be moved to soil.

Seed Set and Transgene Segregation in R1 Generation:

Immature ears from some of the R0 plants were harvested at different times after pollination. The immature embryos from all or some of the kernels were isolated and cultured on MSOD medium supplemented with 200 mg/L paromomycin. The embryos were examined under the fluorescence microscope to determine the number of embryos with GFP expression. In approximately 7 to 10 days, the number of embryos germinated to seedlings was also determined. Other plants were grown to mature and seeds are harvested from individual plants.

### Example 9

Transgenic Plant Analyses

The plants were grown in a greenhouse under appropriate growth conditions as described above. The majority of plants were fully fertile and expressing the appropriate transgene in a 1:1 ratio indicating that the transgene was inherited in a Mendelian fashion. Each plant was examined by one or more of the following methods: a) The GUS histochemical assay (Jefferson, Plant Mol. Biol.Rep., 5:387-405, 1987) using different parts of the plants. b) Southern hybridization analysis (Southern, Mol. Biol., 98:503-517, 1975).

1. A method of obtaining transformable callus tissue comprising:
germinating a mature seed in tissue culture media containing an effective amount of an auxin and an effective amount of a cytokinin to produce a growing seedling containing a nodal section;
isolating the nodal section from the seedling; and
culturing the nodal section on a callus induction media to produce embryogenic callus suitable for transformation.

2. The method of claim 1 in which the auxin is picloram and the cytokinin is BAP.

3. The method of claim 4 in which the picloram concentration is between about 0.5 mg/L and about 20 mg/L.

4. The method of claim 4 in which the BAP concentration is between about 0.1 mg/L and about 10 mg/L.

5. The method of claim 1 in which the tissue culture media is solid.

6. The method of claim 1 in which the nodal section is obtained from the seedling between 3 and 30 days after germination.

7. The method of claim 6 in which the nodal section is obtained from the seedling between 7 and 10 days after germination.

8. The method of claim 1 further comprising the steps of:
transforming the callus with a nucleic acid sequence conferring a selected genetic trait to the transformed callus; and
regenerating a transformed plant from the transformed callus containing the nucleic acid sequence.

9. A method of obtaining transformable callus tissue comprising:
germinating a zygotic embryo in tissue culture media containing an effective amount of an auxin and an effective amount of a cytokinin to produce a growing seedling containing a nodal section;
isolating the nodal section from the seedling; and
culturing the nodal section on a callus induction media to produce embryogenic callus suitable for transformation.

10. The method of claim 9 in which the auxin is picloram and the cytokinin is BAP.

11. The method of claim 10 in which the picloram concentration is between about 0.5 mg/L and about 20 mg/L and the BAP concentration is between about 0.1 mg/L and about 10 mg/L.

12. The method of claim 9 further comprising the steps of:
transforming the callus with a nucleic acid sequence conferring a selected genetic trait to the transformed callus; and
regenerating a transformed plant from the transformed callus to obtain a plant containing the nucleic acid sequence.

13. A method of transforming monocotyledonous plants comprising:
germinating a zygotic embryo from a monocotyledonous plant in tissue culture media containing an effective amount of an auxin and an effective amount of a cytokinin to produce a growing seedling containing a nodal section;
isolating the nodal section from the seedling;
culturing the nodal section in a callus induction media to form an embryogenic callus culture;
transforming the embryogenic callus culture with a nucleic acid sequence conferring a selected genetic trait to the transformed callus;
selecting transformed callus cells; and
regenerating a transformed monocotyledonous plant from the transformed callus to obtain a plant containing the nucleic acid sequence.

14. The method of claim 13 in which the monocotyledonous plant is corn.

15. A method of transforming monocotyledonous plants comprising:
germinating a zygotic embryo from a monocotyledonous plant in tissue culture media containing an effective amount of an auxin and an effective amount of a cytokinin to produce a growing seedling containing a nodal section;
isolating the nodal section from the seedling;
culturing the nodal section in media to form a multiple bud culture;
converting the multiple bud culture to an embryogenic callus culture in callus induction media;
transforming the embryogenic callus culture with a nucleic acid sequence conferring a selected genetic trait to the transformed callus;
selecting transformed callus cells; and
regenerating a transformed monocotyledonous plant from the transformed callus cells to obtain a plant containing the nucleic acid sequence.

16. A method of obtaining transformable callus tissue from a plant comprising:
priming a mature seed;
germinating a mature seed in tissue culture media containing an effective amount of an auxin and an effective amount of a cytokinin to produce a growing seedling containing a nodal section;
isolating the nodal section from the seedling;
culturing the nodal section on callus induction media to produce embryogenic callus.

17. A method of transforming monocotyledonous plants comprising:
priming a mature seed;
germinating the mature seed in tissue culture media containing an effective amount of an auxin and an effective amount of a cytokinin to produce a growing seedling containing a nodal section;
isolating the nodal section from the seedling;
culturing the nodal section on callus induction media to form an embryogenic callus culture;
transforming the embryogenic callus culture with a nucleic acid sequence conferring a selected genetic trait to the transformed callus;
selecting transformed callus cells; and
regenerating a transformed plant from the transformed callus to obtain a plant containing the nucleic acid sequence.
